(19) United States
(12) Patent Application Publication
(10) Pub. No.: US 2009/0247583 A1
(43) Pub. Date: Oct. 1, 2009

(54) PHARMACEUTICAL COMPOSITION COMPRISING, IN COMBINATION, SAREDUTANT AND PAROXETINE

(75) Inventors: Guy GRIEBEL, Fontenay Aux Roses (FR); Caroline COHEN, Paris (FR); Caroline LOUIS, Paris (FR); Lisa Ann ARVANITIS, Kennett Square, PA (US)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/480,052

(22) Filed: Jun. 8, 2009

Related U.S. Application Data
(63) Continuation of application No. 11/830,304, filed on Jul. 30, 2007.

(30) Foreign Application Priority Data
Feb. 7, 2007 (FR) 0700863

(51) Int. Cl. A61K 31/4525; A61P 25/24

(52) U.S. Cl. 514/321

(57) ABSTRACT

A subject-matter of the present invention is pharmaceutical compositions comprising, in combination, at least one active ingredient chosen from (S)-(−)-N-[4-(4-acetamido-4-phenylpiperidin-1-yl)-2-(3,4-dichlorophenyl)butyl]-N-methylbenzamide and pharmaceutically acceptable salts thereof and at least one second active ingredient chosen from trans-(−)-3-[(1,3-benzodioxol-5-yloxy)methyl]-4-(4-fluorophenyl)piperidine and pharmaceutically acceptable salts thereof.

This application is a continuation of U.S. application Ser. No. 11/830,304, filed Jul. 30, 2007, which is incorporated herein by reference in its entirety; which claims the benefit of priority of French Patent Application No. FR07/00,863, filed Feb. 7, 2007.

A subject-matter of the present invention is pharmaceutical compositions comprising, in combination, at least one active ingredient chosen from (S)-(−)-N-[4-(4-acetamido-4-phenylpiperidin-1-yl)-2-(3,4-dichlorophenyl)butyl]-N-methylbenzamide and pharmaceutically acceptable salts thereof and at least one second active ingredient chosen from trans-(−)-3-[(1,3-benzodioxol-5-yloxy)methyl]-4-(4-fluorophenyl)piperidine and pharmaceutically acceptable salts thereof.

(S)-(−)-N-[4-(4-Acetamido-4-phenylpiperidin-1-yl)-2-(3,4-dichlorophenyl)butyl]-N-methylbenzamide, the international nonproprietary name of which is saredutant, of formula:

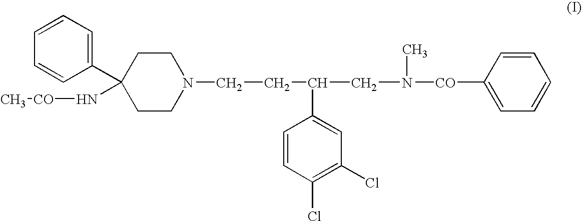

hereinafter referred to as compound A, and its pharmaceutically acceptable salts have been described as nonpeptide antagonists of the NK₂ receptors of neurokinin A (Life Sciences, 1992, 50(15), PL101-PL106) and can be prepared according to Patents EP 0 474 561 or U.S. Pat. No. 5,236,921.

trans-(−)-3-[(1,3-Benzodioxol-5-yloxy)methyl]-4-(4-fluorophenyl)piperidine, the international non-proprietary name of which is paroxetine, of formula:

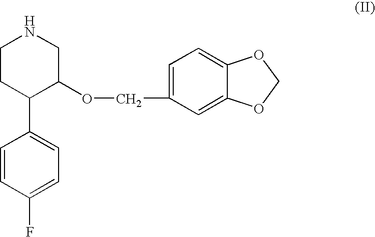

hereinafter referred to as compound B, and its pharmaceutically acceptable salts have been described as selective serotonin reuptake inhibitors (SSRI) and can be prepared according to U.S. Pat. No. 3,912,743 and U.S. Pat. No. 4,007,196.

It has now been found, surprisingly, that the combination of saredutant with paroxetine may significantly enhance the pharmacological effects of each of the compounds used alone, in particular the antidepressant effects.

In another aspect of this invention there is provided a pharmaceutical composition comprising a combination of at least one active ingredient chosen from saredutant and pharmaceutically acceptable salts thereof and at least one second active ingredient chosen from paroxetine and pharmaceutically acceptable salts thereof, further in combination with at least one pharmaceutically acceptable excipient.

In further aspects of this invention there are also provided methods of treatment of various mood disorders and anxiety disorders using the compositions of this invention as specifically disclosed hereinbelow.

The salts are the salts with conventional pharmaceutically acceptable inorganic or organic acids, such as the hydrochloride, the hydrobromide, the sulfate, the hydrogensulfate, the dihydrogenphosphate, the methanesulfonate, the methyl sulfate, the acetate, the oxalate, the maleate, the fumarate, the succinate, the naphthalene-2-sulfonate, the glyconate, the gluconate, the citrate, the isethionate, the benzenesulfonate or the para-toluenesulfonate.

It has now been found, surprisingly, that the combination of saredutant with paroxetine may significantly enhance the pharmacological effects of each of the compounds used alone, in particular the antidepressant effects.

Thus, the pharmaceutical compositions comprising such combination can be of use in the manufacture of medicaments intended for the prevention and treatment of mood disorders, such as major depressive disorder, resistant depressive disorder, dysthymic disorder, depressive disorder not otherwise specified, bipolar I disorder, bipolar II disorder, cyclothymic disorder, bipolar disorder not otherwise specified, mood disorder due to a general medical condition, mood disorder induced by a substance, mood disorder not otherwise specified; anxiety disorders, such as panic attack, agoraphobia, social phobia, obsessive-compulsive disorder, post-traumatic stress condition, acute stress condition, generalized anxiety disorder or anxiety disorder induced by a substance.

In particular, the pharmaceutical compositions comprising such combination can be of use in the manufacture of medicaments intended for the prevention and treatment of a major depressive disorder.

In particular again, the pharmaceutical compositions comprising such combination can be of use in the manufacture of medicaments intended for the treatment of sexual dysfunctions associated with a major depressive disorder.

The term “sexual dysfunctions” is understood to mean any pathology as defined by the American Psychiatric Association—DSM-IV-TR, Diagnostic and Statistical Manual of Mental Disorders, 4th edition, revised text (Washington D.C., 2000), pages 617-654, and which includes disorders of sexual desire (that is to say, the disorder: fall in sexual desire, and the disorder: sexual aversion), disorders of sexual arousal (that is to say, female sexual arousal disorder and male erectile disorder), orgasmic disorders (that is to say, female orgasmic disorder, male orgasmic disorder and premature ejaculation), painful sexual disorders (that is say, dyspareunia and vaginismus), sexual dysfunction due to a general medial condition, sexual dysfunction induced by a substance and sexual dysfunctions not otherwise specified.

Thus, according to one of its aspects, a subject-matter of the present invention is pharmaceutical compositions comprising, in combination, at least one active ingredient chosen from saredutant and pharmaceutically acceptable salts thereof and at least one second active ingredient chosen from paroxetine and pharmaceutically acceptable salts thereof, and also at least one pharmaceutically acceptable excipient.

According to another of its aspects, a subject-matter of the present invention is the combination of at least one active ingredient chosen from saredutant and pharmaceutically acceptable salts thereof and at least one second active ingredient chosen from paroxetine and pharmaceutically acceptable salts thereof.

According to another of its aspects, a subject-matter of the present invention is the use of a pharmaceutical composition comprising, in combination, at least one active ingredient chosen from saredutant and pharmaceutically acceptable salts thereof and at least one second active ingredient chosen from paroxetine and pharmaceutically acceptable salts thereof, in the manufacture of medicaments intended for the prevention and treatment of mood disorders chosen from major depressive disorder, resistant depressive disorder, dysthymic disorder, depressive disorder not otherwise specified, bipolar I disorder, bipolar II disorder, cyclothymic disorder, bipolar disorder not otherwise specified, mood disorder due to a general medical condition, mood disorder induced by a substance, mood disorder not otherwise specified; anxiety disorders, chosen from panic attack, agoraphobia, social phobia, obsessive-compulsive disorder, post-traumatic stress condition, acute stress condition, generalized anxiety disorder or anxiety disorder induced by a substance.

In particular, a subject-matter of the present invention is the use of a pharmaceutical composition comprising, in combination, at least one active ingredient chosen from saredutant and pharmaceutically acceptable salts thereof and at least one second active ingredient chosen from paroxetine and pharmaceutically acceptable salts thereof, in the manufacture of medicaments intended for the prevention and the treatment of a major depressive disorder.

In particular again, a subject-matter of the present invention is the use of a pharmaceutical composition comprising, in combination, at least one active ingredient chosen from saredutant and pharmaceutically acceptable salts thereof and at least one second active ingredient chosen from paroxetine and pharmaceutically acceptable salts thereof, in the manufacture of medicaments intended for the treatment of sexual dysfunctions associated with a major depressive disorder.

According to another of its aspects, a subject-matter of the present invention is the use of the combination of at least one active ingredient chosen from saredutant and pharmaceutically acceptable salts thereof and at least one second active ingredient chosen from paroxetine and pharmaceutically acceptable salts thereof, in the manufacture of medicaments intended for the prevention and treatment of mood disorders chosen from major depressive disorder, resistant depressive disorder, dysthymic disorder, depressive disorder not otherwise specified, bipolar I disorder, bipolar II disorder, cyclothymic disorder, bipolar disorder not otherwise specified, mood disorder due to a general medical condition, mood disorder induced by a substance, mood disorder not otherwise specified; anxiety disorders, chosen from panic attack, agoraphobia, social phobia, obsessive-compulsive disorder, post-traumatic stress condition, acute stress condition, generalized anxiety disorder or anxiety disorder induced by a substance.

The excipients are chosen, according to the pharmaceutical form and the method of administration desired, from the usual excipients which are known to a person skilled in the art.

In the pharmaceutical compositions of the present invention for oral, sublingual, subcutaneous, intramuscular, intravenous, topical, local, intratracheal, intranasal, transdermal or rectal administration, the active principles can be administered in unit administration form, as a mixture with conventional pharmaceutical excipients, to animals and human beings for the prevention or treatment of the above disorders or diseases.

The appropriate unit administration forms comprise oral forms, such as tablets, soft or hard gelatin capsules, powders, granules and oral solutions or suspensions, sublingual, buccal, intratracheal, intraocular or intranasal administration forms, forms for administration by inhalation, topical, transdermal, subcutaneous, intramuscular or intravenous administration forms, rectal administration forms and implants. For topical application, the compounds according to the invention can be used in creams, gels, ointments or lotions.

In the pharmaceutical compositions of the present invention, the active principle or the active principles are generally formulated in dosage units containing from 2.5 to 500 mg, advantageously from 10 to 250 mg and preferably from 10 to 150 mg of the said active principle per dosage unit for daily administrations. There may be particular case in which higher or lower dosages are appropriate; such dosages do not depart from the context of the invention. According to the usual practice, the dosage that is appropriate for each patient is determined by the doctor according to the mode of administration and the weight and response of the patient.

According to another aspect of the invention, the compound A and the compound B according to the invention can be administered simultaneously, separately or spread out over time.

The term “simultaneous” is understood to mean the administration of the compounds of the composition according to the invention comprise within one and the same pharmaceutical form.

The term “separate” is understood to mean the administration, at the same time, of the two compounds of the composition according to the invention, each comprised within a separate pharmaceutical form.

The term “spread out over time” is understood to mean the successive administration of the first compound of the composition according to the invention, comprised within a pharmaceutical form, and then of the second compound of the composition according to the invention, comprised within a separate pharmaceutical form.

In the case of this “spread out over time”, the period of time elapsed between the administration of the first compound of the composition according to the invention and the administration of the second compound of the same composition according to the invention generally does not exceed 24 hours.

The unit pharmaceutical forms comprising either just one of the constituent compounds of the composition according to the invention or the combination of the 2 compounds which can be employed in the various types of use described above may, for example, be appropriate for oral, nasal, parenteral or transdermal administration.

Consequently, in the case of a “separate” use and of a use “spread out over time”, two separate pharmaceutical forms may be intended for the same route of administration or for a different route of administration (oral and transdermal or oral and nasal or parenteral and transdermal, and the like).

The invention thus also relates to a kit comprising the compound A and the compound B according to the invention in which the said compound A and the compound B according to the invention are in separate compartments and in similar or different packagings and are intended to be administered simultaneously, separately or spread out over time.

Specifically and without implied limitation, the effects of a combination according to the invention of the compound A and of paroxetine are studied during clinical studies.

### EXAMPLE 1

The effects of a combination according to the invention of saredutant with paroxetine are evaluated during multicentric randomized double-blind clinical studies lasting eight weeks in which a group receiving a fixed dose of either 30 mg or 100 mg saredutant once daily in combination with a fixed dose of 20 mg paroxetine once daily is compared with a group receiving saredutant placebo in combination with a fixed dose of paroxetine and with another group receiving saredutant placebo in combination with paroxetine placebo.

These clinical studies are carried out on adult male or female patients exhibiting a major depressive disorder as defined by the criteria of the Diagnostic and Statistical Manual of Mental Disorders, 4th edition, revised text (DSM-IV-TR), and confirmed by the criteria of the Mini International Neuropsychiatric Interview (MINI), Lecrubier Y. et al., Eur. Psychiatr., 1997, 12, 224-231.

The change in the total score on the Hamilton Depression Rating Scale comprising 17 items (HAM-D) between the final visit (expected on the 56th day) and the first visit (before the beginning of the treatment) is measured with respect to the placebo group. The Hamilton Depression Rating Scale is defined by Hamilton M., J. Neurol. Neurosurg. Psychiatry, 1960, 23, 56-62.

The change in the total score of the CSFQ comprising 14 items (Changes in Sexual Functioning Questionnaire) between the final visit (expected on the 56th day) and the first visit (before the beginning of the treatment) is also measured with respect to the placebo group. The CSFQ is defined by Clayton A H et al., Psychopharmacol. Bull., 1997, 33, 731-745.

### EXAMPLE 2

A pharmaceutical composition in accordance with this invention in the form of a capsule comprising 30 mg of saredutant was prepared including the following pharmaceutically acceptable excipients.

| Saredutant (expressed as base) | 30.0 | mg |
|---|---|---|
| Lactose monohydrate (200 mesh) | QSP 400.0 | mg |
| Croscarmellose sodium | 8.0 | mg |
| Magnesium stearate | 4.0 | mg |
| Purified water* | QS | |
| Size-0 opaque hard capsule, filled with | 400.0 | mg |

*drying evaporated after moist grainy effect.

### EXAMPLE 3

A pharmaceutical composition in accordance with this invention in the form of a capsule comprising 100 mg of saredutant was prepared including the following pharmaceutically acceptable excipients.

| Saredutant (expressed as base) | 100.0 | mg |
|---|---|---|
| Lactose monohydrate (200 mesh) | QSP 400.0 | mg |
| Croscarmellose sodium | 8.0 | mg |
| Magnesium stearate | 4.0 | mg |
| Purified water* | QS | |
| Size-0 opaque hard capsule, filled with | 400.0 | mg |

*drying evaporated after moist grainy effect.

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

What is claimed is:

1. A combination comprising at least one active ingredient chosen from saredutant and pharmaceutically acceptable salts thereof and at least one second active ingredient chosen from paroxetine and pharmaceutically acceptable salts thereof.

2. The combination according to claim 1, wherein said at least one active ingredient and said at least one second active ingredient are administered simultaneously, separately, or spread out over time.

3. The combination according to claim 2, wherein said at least one active ingredient and said at least one second active ingredient are administered simultaneously.

4. The combination according to claim 2, wherein said at least one active ingredient and said at least one second active ingredient are administered separately.

5. The combination according to claim 2, wherein said at least one active ingredient and said at least one second active ingredient are administered spread out over time.

6. A pharmaceutical composition comprising at least one active ingredient chosen from saredutant and pharmaceutically acceptable salts thereof and at least one second active ingredient chosen from paroxetine and pharmaceutically acceptable salts thereof in combination with at least one pharmaceutically acceptable excipient.

7. The composition according to claim 6, wherein said at least one active ingredient and said at least one second active ingredient are administered simultaneously, separately, or spread out over time.

8. The composition according to claim 7, wherein said at least one active ingredient and said at least one second active ingredient are administered simultaneously.

9. The composition according to claim 7, wherein said at least one active ingredient and said at least one second active ingredient are administered separately.

10. The composition according to claim 7, wherein said at least one active ingredient and said at least one second active ingredient are administered spread out over time.

11. A method of treatment of a mood disorder in a patient comprising administering to the patient a therapeutically effective amount of at least one active ingredient chosen from saredutant and pharmaceutically acceptable salts thereof and at least one second active ingredient chosen from paroxetine and pharmaceutically acceptable salts thereof optionally in combination with one or more pharmaceutically acceptable excipients.

12. The method according to claim 11, wherein said at least one active ingredient and said at least one second active ingredient are administered simultaneously, separately or spread out over time.

13. The method according to claim 12, wherein said at least one active ingredient and said at least one second active ingredient are administered simultaneously.

14. The method according to claim 12, wherein said at least one active ingredient and said at least one second active ingredient are administered separately.

15. The method according to claim 12, wherein said at least one active ingredient and said at least one second active ingredient are administered spread out over time.

16. The method according to claim 11, wherein the mood disorder is major depressive disorder.

17. The method according to claim 16, wherein the major depressive disorder is sexual dysfunctions associated with a major depressive disorder.

18. The method according to claim 11, wherein the mood disorder is resistant depressive disorder.

19. The method according to claim 11, wherein the mood disorder is dysthymic disorder.

20. The method according to claim 11, wherein the mood disorder is depressive disorder not otherwise specified.
